(19) United States
(12) Patent Application Publication
(10) Pub. No.: US 2023/0036073 A1
(43) Pub. Date: Feb. 2, 2023

(54) ENHANCED ACOUSTIC PARTICLE PROCESSING WITH SEEDING PARTICLES

(71) Applicant: FloDesign Sonics, Inc., West Springfield, MA (US)

(72) Inventors: Benjamin Ross-Johnsrud, Burlington, MA (US); Kedar C. Chitale, Burlington, MA (US); Bart Lipkens, Burlington, MA (US); Rui Tostoes, Burlington, MA (US)

(21) Appl. No.: 17/759,009

(22) PCT Filed: Jan. 19, 2021
(86) PCT No.: PCT/US2021/013926

§ 371 Date: Jul. 18, 2022

Related U.S. Application Data
(60) Provisional application No. 62/962,860, filed on Jan. 17, 2020.

(51) Int. Cl. B06B 1/02; B06B 3/04; B01D 43/00; H01L 41/09; B06B 1/06

(52) U.S. Cl. CPC B06B 1/0292; B06B 3/04; B01D 43/00; H01L 41/09; B06B 1/0607; B06B 1/067; B06B 2201/70

(57) ABSTRACT

Acoustic forces in an acoustic field can be increased via introduction of “seeding particles” with higher or similar contrast factor and/or size relative to the particles targeted for retention in the acoustic field. This feature may be implemented in an acoustic concentration device or an acoustic separation device. Increases in acoustic forces lead to better particle retention and can permit increased flow rates through an acoustic particle processing device.

### CROSS-REFERENCE TO RELATED APPLICATIONS

The present application claims the benefit of priority of U.S. Provisional Application No. 62/962,860, filed Jan. 17, 2020, the entire contents of which is incorporated herein by reference.

### BACKGROUND

In some implementations, acoustophoresis is the manipulation of particles or fluids using acoustic waves. Acoustic waves can exert forces on particles in a fluid when there is a differential in density and/or compressibility between the particles and fluid, otherwise known as the acoustic contrast factor. For an acoustic standing wave, the pressure profile includes areas of local minimum pressure amplitudes at standing wave nodes and local maxima at standing wave anti-nodes. Depending on the density and/or compressibility, the particles become trapped at the nodes or anti-nodes of the standing wave. Generally, the higher the frequency of the standing wave, the smaller the particles that can be trapped due the pressure profile of the standing wave.

Manipulation of materials that have different acoustic contrast factors (a combination of density and the speed of sound through the material) has been demonstrated at the MEMS (micro electrical mechanical systems) scale. At the MEMS scale, conventional acoustic systems rely on using half or quarter wavelength acoustic chambers, which at frequencies of a few megahertz are typically less than a millimeter in thickness, and operate at very low flow rates (e.g., μL/min). Such systems are not scalable since they benefit from extremely low Reynolds number, laminar flow operation, and obtain minimal fluid dynamic optimization.

At the macro-scale, planar acoustic standing waves have been used for acoustic particle manipulation. However, a single planar acoustic wave tends to trap the particles in planes located at nodes or anti-nodes the planar standing wave. The particles do not tend to cluster, and remain suspended in the fluid in which they are entrained until the planar acoustic wave is removed (turned off). Particle manipulation using a planar acoustic standing wave is thus not continuous. Also, the amount of power that is used to generate the acoustic planar standing wave tends to heat the fluid through waste energy.

### SUMMARY

The present disclosure relates, in various embodiments, to macro-scale acoustic devices with improved fluid dynamics that can be used to improve the manipulation of particles (e.g. cells) in a particle/fluid mixture. In some example implementations, an ultrasonic transducer that includes a piezoelectric material is coupled to a chamber to permit an acoustic wave to be generated within the chamber. The piezoelectric material is configured to be excited to launch an acoustic wave in the chamber. According to some examples, the piezoelectric material is constructed to be capable of deforming to permit being excited in a higher order mode shape to generate distinct and different acoustic waves in multiple directions in the chamber.

According to some examples, acoustic forces in an acoustic field can be increased via introduction of “seeding particles” with higher or similar contrast factor and/or size relative to the particles targeted for retention in the acoustic field. This feature may be implemented in an acoustic concentration device or an acoustic separation device. Increases in acoustic forces lead to better particle retention and can permit increased flow rates through an acoustic particle processing device.

### BRIEF DESCRIPTION OF THE DRAWINGS

The following is a brief description of the drawings, which are presented for the purposes of illustrating the exemplary embodiments disclosed herein and not for the purposes of limiting the same.

FIG. 1 is a cross-sectional diagram of a conventional ultrasonic transducer.

FIG. 2 is a cross-sectional diagram of an ultrasonic transducer of the present disclosure. An air gap is present within the transducer, and no backing layer or wear plate is present.

FIG. 3 is a cross-sectional diagram of an ultrasonic transducer of the present disclosure. An air gap is present within the transducer, and a backing layer and wear plate are present.

FIG. 4 is a graph showing the relationship of the acoustic radiation force, gravity/buoyancy force, and Stokes' drag force to particle size. The horizontal axis is in microns (μm) and the vertical axis is in Newtons (N).

FIG. 5 is a graph of electrical impedance amplitude versus frequency for a square transducer driven at different frequencies.

FIG. 6 illustrates the trapping line configurations for seven peak amplitudes of an ultrasonic transducer of the present disclosure.

FIG. 7 is a perspective view illustrating a separator of the present disclosure. The fluid flow direction and the trapping lines are shown.

FIG. 8 is a view from the fluid inlet along the fluid flow direction (arrow 814) of FIG. 7, showing the trapping nodes of the standing wave where particles would be captured.

FIG. 9 is a view taken through the transducer face at the trapping line configurations, along arrow 816 as shown in FIG. 7.

FIG. 10 is a diagram of an acoustophoresis system employed with a bioreactor.

FIGS. 11, 12 and 13 are diagrams of an acoustophoresis device for co-locating particle clusters and reagents.

FIG. 14 is a graph of a seeding particle retention in an acoustic wave.

### DETAILED DESCRIPTION

The present disclosure may be understood more readily by reference to the following detailed description of desired embodiments and the examples included therein. In the following specification and the claims which follow, reference will be made to a number of terms which shall be defined to have the following meanings.

Although specific terms are used in the following description for the sake of clarity, these terms are intended to refer only to the particular structure of the embodiments selected for illustration in the drawings, and are not intended to define or limit the scope of the disclosure. In the drawings and the following description below, it is to be understood that like numeric designations refer to components of like function.

The singular forms “a,” “an,” and “the” include plural referents unless the context clearly dictates otherwise.

The term “comprising” is used herein as requiring the presence of the named component and allowing the presence of other components. The term “comprising” should be construed to include the term “consisting of”, which allows the presence of only the named component, along with any impurities that might result from the manufacture of the named component.

Numerical values should be understood to include numerical values which are the same when reduced to the same number of significant figures and numerical values which differ from the stated value by less than the experimental error of conventional measurement technique of the type described in the present application to determine the value.

All ranges disclosed herein are inclusive of the recited endpoint and independently combinable (for example, the range of “from 2 grams to 10 grams” is inclusive of the endpoints, 2 grams and 10 grams, and all the intermediate values). The endpoints of the ranges and any values disclosed herein are not limited to the precise range or value; they are sufficiently imprecise to include values approximating these ranges and/or values.

The modifier “about” used in connection with a quantity is inclusive of the stated value and has the meaning dictated by the context. When used in the context of a range, the modifier “about” should also be considered as disclosing the range defined by the absolute values of the two endpoints. For example, the range of “from about 2 to about 10” also discloses the range “from 2 to 10.” The term “about” may refer to plus or minus 10% of the indicated number. For example, “about 10%” may indicate a range of 9% to 11%, and “about 1” may mean from 0.9-1.1.

It should be noted that many of the terms used herein are relative terms. For example, the terms “upper” and “lower” are relative to each other in location, e.g. an upper component is located at a higher elevation than a lower component in a given orientation, but these terms can change if the device orientation is changed. The terms “inlet” and “outlet” are relative to a fluid flowing through them with respect to a given structure, e.g. a fluid flows through the inlet into the structure and flows through the outlet out of the structure. The terms “upstream” and “downstream” are relative to the direction in which a fluid flows through various components, i.e. the flow fluids through an upstream component prior to flowing through the downstream component. It should be noted that in a loop, a first component can be described as being both upstream of and downstream of a second component.

The terms “horizontal” and “vertical” are used to indicate direction relative to an absolute reference, i.e. ground level. However, these terms should not be construed to require structures to be absolutely parallel or absolutely perpendicular to each other. For example, a first vertical structure and a second vertical structure are not necessarily parallel to each other. The terms “top” and “bottom” are used to refer to surfaces where the top is always higher than the bottom relative to an absolute reference, i.e. the surface of the earth. The terms “upwards” and “downwards” are also relative to an absolute reference; upwards is always against the gravity of the earth.

The term “parallel” should be construed in its lay sense of two surfaces that maintain a generally constant distance between them, and not in the strict mathematical sense that such surfaces will never intersect when extended to infinity.

The present application refers to “the same order of magnitude.” Two numbers are of the same order of magnitude if the quotient of the larger number divided by the smaller number is a value of at least 1 and less than 10.

The term “virus” refers to an infectious agent that can only replicate inside another living cell, and otherwise exists in the form of a virion formed from a capsid that surrounds and contains DNA or RNA, and in some cases a lipid envelope surrounding the capsid.

The term “crystal” refers to a single crystal or polycrystalline material that is composed of a piezoelectric material.

The acoustophoretic devices of the present disclosure are suitable for cell processing for use in immunotherapy, such as cell therapy, including CAR-T therapy. The acoustophoretic devices are designed to create an acoustic field that that includes locales with a pressure and/or acoustic radiation force (ARF) that is greater than the combined effects of fluid drag and gravity/buoyancy, and is therefore able to trap (i.e., hold stationary) the suspended phase (i.e. cells). As a result, in the present devices, the acoustic field acts as a filter that prevents targeted particles (e.g., biological cells) from passing. The trapping capability of the acoustic field may be varied as desired, for example, by varying the flow rate of the fluid, the power delivered to the ultrasonic transducer, the shape and magnitude of the acoustic field, and the shape of the acoustophoretic device. The acoustophoretic devices of the present disclosure have the ability to create acoustic fields that can trap particles in flow fields with flow rates greater than 1 mL/minute.

As used herein, particles may be solid, liquid (droplets) or gas (bubbles), or multi-phase, e.g., a solid shell with a liquid or gas core. The particles may be rigid or flexible, solid or hollow or consist of layered materials. Particles may be biomaterials, including but not limited to cells, viruses, vesicles or biomolecules, e.g., mAbs or proteins. Particles may be complexes, such as, for example, bead-cell complexes or clusters of particles.

The scattering of the acoustic field off the particles results in a three-dimensional acoustic radiation force, which acts as a three-dimensional trapping field. The acoustic radiation force is proportional to the particle volume (e.g. the cube of the radius) when the particle is small relative to the wavelength. The acoustic radiation force is proportional to frequency of the ultrasonic transducer operation and the acoustic contrast factor. The acoustic radiation force scales with acoustic energy (e.g. the square of the acoustic pressure amplitude). When the acoustic radiation force exerted on the particles is stronger than the combined effect of fluid drag force and gravitational/buoyancy force, the particles are trapped within the acoustic field. The action of the acoustic forces on the trapped particles results in formation of tightly-packed clusters through concentration, clustering, clumping, agglomeration and/or coalescence of particles. In some implementations, the particle clustering produces effective particle sizes that meet a threshold where the acoustic radiation force is no longer dominant, causing the cluster to exit the acoustic field. Particulate settling occurs through enhanced gravitational force for particles heavier (denser) than the host fluid. Particulate rising occurs through enhanced buoyancy for particles lighter (less dense) than the host fluid.

Most biological cell types present a higher density and lower compressibility than the medium in which they are suspended, so that the acoustic contrast factor between the cells and the medium has a positive value. As a result, the pressure and/or acoustic radiation force drives the positive acoustic contrast factor cells towards the locales of low net pressure and/or acoustic radiation force. Cells or particles with a negative acoustic contrast factor are driven to locales of high net pressure and/or acoustic radiation force within the acoustic field. The pressure and/or acoustic radiation force can be dominant dominant over fluid drag force and gravitational/buoyancy force for particle sizes in a range of about 10 μm or greater. For small cells or emulsions the drag force F_{D} can be expressed as:

$${\vec{F}}_{D} = 4 π {μ}_{f} {R}_{p} ( {\vec{U}}_{f} - {\vec{U}}_{p} ) [ \frac{1 + \frac{3}{2} \hat{μ}}{1 + \hat{μ}} ]$$

where U_{f} and Uₚ are the fluid and cell velocity, Rₚ is the particle radius, μ_{f} and μₚ are the dynamic viscosity of the fluid and the cells, and \hat{μ}=μₚ/μ_{f} is the ratio of dynamic viscosities. The buoyancy force F_{B} is expressed as:

$${F}_{B} = \frac{4}{3} π {R}_{p}^{3} ( {ρ}_{f} - {ρ}_{p} ) .$$

For a cell to be trapped in the acoustic field, the force balance or sum of the force vectors on the cell may be assumed to be zero, and therefore an expression for acoustic radiation force F_{ARF} can be found, which is given by:

F_{ARF}=F_{D}+F_{B}.

For a cell of known size and material property, and for a given flow rate, this equation can be used to estimate the magnitude of the acoustic radiation force. One theoretical model that is used to calculate the acoustic radiation force is based on the formulation developed by Gor′kov. The primary acoustic radiation force F_{A} is defined as a function of a field potential U, F_{A}=−Λ(U), where the field potential U is defined as

$$U = {V}_{0} [ \frac{〈 {p}^{2} 〉}{2 {ρ}_{f} {c}_{f}^{2}} {f}_{1} - \frac{3 {ρ}_{f} 〈 {u}^{2} 〉}{4} {f}_{2} ] ,$$

and f₁ and f₂ are the monopole and dipole contributions defined by

$${f}_{1} = 1 - \frac{1}{{Λσ}^{2}} , \qquad {f}_{2} = \frac{2 ( Λ - 1 )}{2 Λ + 1} ,$$

where ρ is the acoustic pressure, u is the fluid particle velocity, Λ is the ratio of cell density ρₚ to fluid density ρ_{f}, σ is the ratio of cell sound speed cₚ to fluid sound speed c_{f}, V_{D} is the volume of the cell, and < > indicates time averaging over the period of the wave.

Gork′ov's model is for a single particle in a standing wave and is limited to particle sizes that are small with respect to the wavelength of the sound fields in the fluid and the particle. It also does not take into account the effect of viscosity of the fluid and the particle on the radiation force. As a result, this model is not used for the macro-scale ultrasonic separators discussed herein since particle clusters can grow quite large. A more complex and complete model for acoustic radiation forces that is not limited by particle size was therefore used. The models that were implemented are based on the theoretical work of Yurii Ilinskii and Evgenia Zabolotskaya as described in AIP Conference Proceedings, Vol. 1474-1, pp. 255-258 (2012). These models also include the effect of fluid and particle viscosity, and therefore are a more accurate calculation of the acoustic radiation force.

The total acoustic radiation force generated by the ultrasonic transducers of the present disclosure is significant and is sufficient to overcome the fluid drag force. The acoustic radiation force can thus be used to retain cells within the acoustic field while the fluid in which the cells are entrained flows through the acoustic field. The pressure and/or acoustic radiation force applied to the trapped particles results in formation of tightly packed clusters through concentration, agglomeration and/or coalescence of particles. Relatively large solids of one material can thus be separated from smaller particles of a different material, the same material, and/or the host fluid. Alternatively, or in addition, particles can be provided to, trapped and retained in the acoustic field for further processing. For example, reactions on the particles can be performed within the acoustic field, such as, for example, by providing reagents to cause cell reactions for cells retained in an acoustic field.

In some implementations an ultrasonic transducer is configured with piezoelectric material that deforms when excited by an electrical signal. The electrical signal can be provided at a frequency that causes the piezoelectric material to deform to obtain fluctuating peaks and valleys on a surface of the piezoelectric material, referred to and defined herein as multimode shape or multimode operation. The deformation of the piezoelectric material generates different and distinct acoustic waves in multiple directions, referred to and defined herein as multi-directional waves. The multi-directional waves generate acoustic radiation forces in the axial direction, e.g., in the direction of wave propagation, and in the lateral direction, e.g., in the direction transverse to the direction of wave propagation. The multi-directional acoustic waves are launched in a volume of the fluid, meaning that they are bulk acoustic waves. Bulk acoustic waves propagate through a volume of a material, and thus operate on a different principle than surface acoustic waves, which propagate on a surface of a material and do not penetrate significantly into a volume of the material.

The multi-directional acoustic waves are used with fluid containers or chambers that house flowing or non-flowing fluids. The size of the container accommodates a significant number of wavelengths of the multi-directional acoustic waves, for example, 10 or greater wavelengths, or 20 or greater wavelengths. A reflector may be provided to the container on an opposite side from that of the ultrasonic transducer, resulting in a multi-directional acoustic standing wave at certain resonance frequencies. As the mixture flows through the acoustic chamber, particles in suspension experience a strong axial force component in the direction of propagation of the standing wave. Since this acoustic force is perpendicular to the flow direction and the drag force, the particles are rapidly moved to pressure nodal planes or anti-nodal planes, depending on the contrast factor of the particle. Multi-directional acoustic standing waves can produce significant lateral acoustic radiation force components that are transverse to the direction of wave propagation. The lateral acoustic radiation force can cause the concentrated particles to be moved towards the center of each planar node, resulting in agglomeration or clumping of material at a point, rather than a plane. The lateral acoustic radiation force component overcomes fluid drag, which permits clumps of particles to continually grow and then drop out of the mixture due to gravity. A drop in drag per particle as the particle cluster increases in size and drop in acoustic radiation force per particle as the particle cluster grows in size, may be considered together or independently in the operation of the acoustic separator device. In at least some examples in the present disclosure, the lateral force component and the axial force component of the multi-directional acoustic standing wave are of the same order of magnitude. In this regard, it is noted that in a multi-directional acoustic standing wave, the axial force may have a different value than the lateral force, e.g. be weaker or stronger, or may be equal or equivalent, but the lateral force of a multi-directional acoustic standing wave is greater than the lateral force of a planar standing wave, sometimes by two orders of magnitude or more.

A multi-directional acoustic wave can be reflected by a reflector to form a multi-directional acoustic standing wave, which can be employed to manipulate particles in a number of applications, including filtering or separation. The standing waves can be used to trap the cells and cell debris present in the cell culture media. The cells and cell debris, having a positive contrast factor, move to the nodes (as opposed to the anti-nodes) of the standing wave. As the cells and cell debris agglomerate at the nodes of the standing wave, there is also a physical scrubbing of the cell culture media that occurs whereby more cells are trapped as they come in contact with the cells that are already held within the standing wave. This trapping generally separates the cells and cellular debris from the cell culture media. When the cells in the standing wave agglomerate to the extent where the mass is no longer able to be held by the acoustic wave, the aggregated cells and cellular debris that have been trapped can fall out of the fluid stream through gravity, and can be collected separately. To aid this gravitational settling of the cells and cell debris, the standing wave may be interrupted to allow all of the cells to fall out of the fluid stream that is being filtered. This process can be useful for dewatering. The expressed biomolecules may have been removed beforehand, or remain in the fluid stream (i.e. cell culture medium).

In the present disclosure, a perfusion bioreactor can also be used to generate cells that can subsequently be used for various applications, including cell therapy. In this type of process, the biological cells to be used in the cell therapy are cultured in the bioreactor and expanded (i.e. to increase the number of cells in the bioreactor through cell reproduction). These cells may be lymphocytes such as T cells (e.g., regulatory T-cells (Tregs), Jurkat T-cells), B cells, or NK cells; their precursors, such as peripheral blood mononuclear cells (PBMCs); and the like. In the perfusion bioreactor, the cell culture media (aka host fluid), containing some cells, is sent from the bioreactor to a filtering device that produces an acoustic wave and an attendant acoustic field. A majority of the cells are trapped and held in the acoustic field, while the remaining host fluid and other cells in the host fluid are returned to the bioreactor. As the quantity of trapped cells increases, they form larger clusters that will fall out of the acoustic field at a critical size due to gravity forces. The clusters can fall into a concentrate outlet outside a region of the acoustic field, such as below the acoustic field, from which the cells can be recovered for use in cell therapy. Only a small portion of the cells are trapped and removed from the bioreactor via the concentrate outlet, and the remainder continue to reproduce in the bioreactor, allowing for continuous production and recovery of the desired cells.

In some applications, the acoustophoretic devices of the present disclosure can act as a cell retention device. The systems described herein operate over a range of cell recirculation rates, efficiently retain cells over a range of perfusion (or media removal) rates, and can be tuned to fully retain or selectively pass some percentage of cells through fluid flow rate, transducer power or frequency manipulation. Power and flow rates can all be monitored and used as feedback in an automated control system.

The cells of interest may also be held in the flow chamber of the external filtering device with an acoustic field so that other moieties may be introduced in close proximity to and for the purpose of changing the target cells. An example of such an operation includes the trapping of T cells and the subsequent introduction of modified lentivirus materials with a specific gene splice such that the lentivirus with a specific gene splice will transfect the T cell and generate a chimeric antigen receptor T cell also known as a CAR-T cell.

The acoustophoresis devices disclosed herein may be used for processing bioreactor materials. A bioreactor can be the source of fluid that is provided to the acoustophoresis device, where the fluid is a mixture of a host/primary fluid (e.g. water, cell culture media) and a secondary particulate. The secondary particulate can include cells and expressed materials such as biomolecules (e.g. recombinant proteins or monoclonal antibodies or viruses). The acoustophoresis devices can be used to concentrate larger particles, such as cells, from the mixture so that the particles are separated from the mixture. The particles and the depleted mixture can be removed separately from the acoustophoresis device, e.g., via different outlets. Concentrated cells can be removed from one outlet, for example, while fluid that is depleted of cells can be removed from another outlet. Either of these two outputs can be recycled to the bioreactor, and/or can be delivered to separate receptacles, such as a product receptacle or a waste receptacle, and/or can be delivered to additional devices for further processing.

Desirably, flow rates through the devices of the present disclosure can be in a range of from about 1 mL/min to about 800 mL/min. In alternate units, these flow rates may be about 0.005 mL/min per cm² of cross-sectional area of the acoustic chamber, or about 4.5 mL/min/cm².

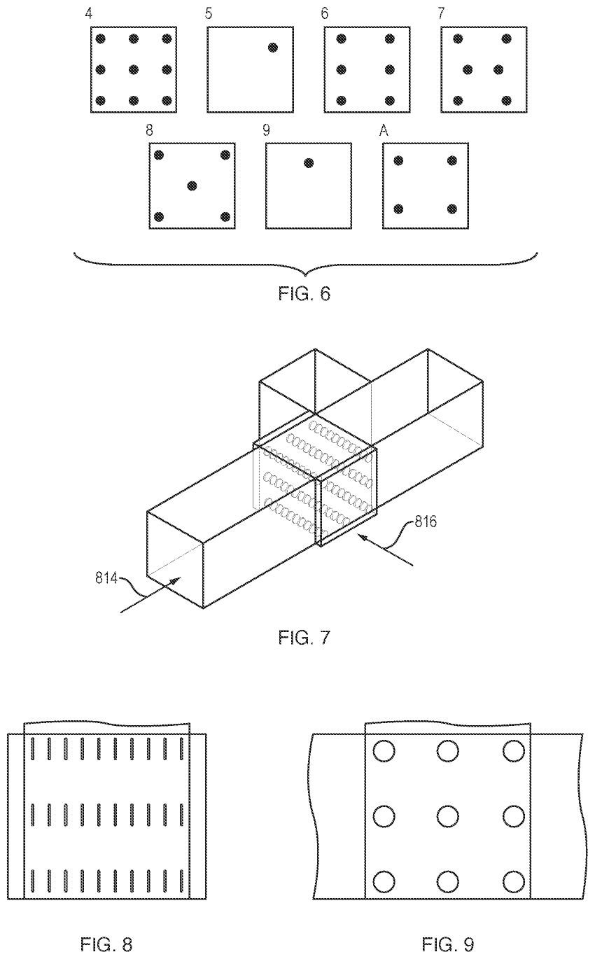

The multi-directional acoustic wave used for particle collection is obtained by driving the deformable piezoelectric material of an ultrasonic transducer at a frequency that excites the piezoelectric material into a high order mode shape, or multimode shape. A piezoelectric crystal can be specifically designed to deform in a multimode shape at designed frequencies, allowing for generation of multi-directional acoustic waves. The multi-directional acoustic wave may be generated by distinct modes of the piezoelectric crystal such as a 3×3 mode, illustrated as number 4 in FIG. 6. A multitude of multi-directional acoustic waves may also be generated by allowing the piezoelectric crystal to vibrate through many different mode shapes. Thus, the crystal would excite multiple modes such as a 0x0 mode (i.e. a piston mode) to a 1×1, 2×2, 1×3, 3×1, 3×3, and other higher order modes and then cycle back through the lower modes of the crystal (not necessarily in straight order). This switching or dithering of the crystal between modes allows for various multi-directional wave shapes, along with a single piston mode shape to be generated over a designated time.

The transducers use a piezoelectric crystal, usually made of PZT-8 (lead zirconate titanate). Some examples of such crystals include 1 inch square and 1 inch diameter crystals that and a nominal 2 MHz resonance frequency, and may also be of a larger size. Each ultrasonic transducer module can have only one crystal, or can have multiple crystals that each act as a separate ultrasonic transducer and are either controlled by one or multiple drivers. The crystals can be square, rectangular, irregular polygon, or generally of any arbitrary shape. The transducer(s) is/are used to create a pressure field that generates forces of the same order of magnitude both orthogonal to the acoustic wave direction (lateral) and in the acoustic wave direction (axial).

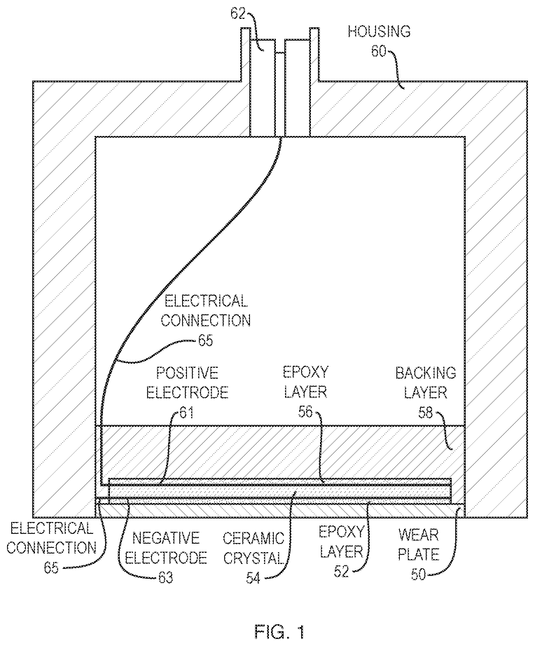

FIG. 1 is a cross-sectional diagram of a conventional ultrasonic transducer. This transducer has a wear plate 50 at a bottom end, epoxy layer 52, ceramic crystal 54 (made of, e.g. PZT), an epoxy layer 56, and a backing layer 58. On either side of the ceramic crystal, there is an electrode: a positive electrode 61 and a negative electrode 63. The epoxy layer 56 attaches backing layer 58 to the crystal 54. The entire assembly is contained in a housing 60 which may be made out of, for example, aluminum. An electrical adapter 62 provides connection for wires to pass through the housing and connect to leads (not shown) which attach to the crystal 54. Typically, backing layers are designed to add damping and to create a broadband transducer with uniform displacement across a wide range of frequency and are designed to suppress excitation at particular vibrational eigen-modes. Wear plates are usually designed as impedance transformers to better match the characteristic impedance of the medium into which the transducer radiates.

According to some example implementations, a potting material, such as epoxy, is used to attach the piezoelectric material to the housing. An adhesive-backed film, made for example from a polyetheretherketone (PEEK), is attached to the exterior surface of the piezoelectric material and the housing. This film can act as a wear layer. The wear layer generally has a thickness of a half wavelength or less (e.g., 0.050 inches). Additional features of the ultrasonic transducer(s) used in the present devices will be explained in greater detail herein.

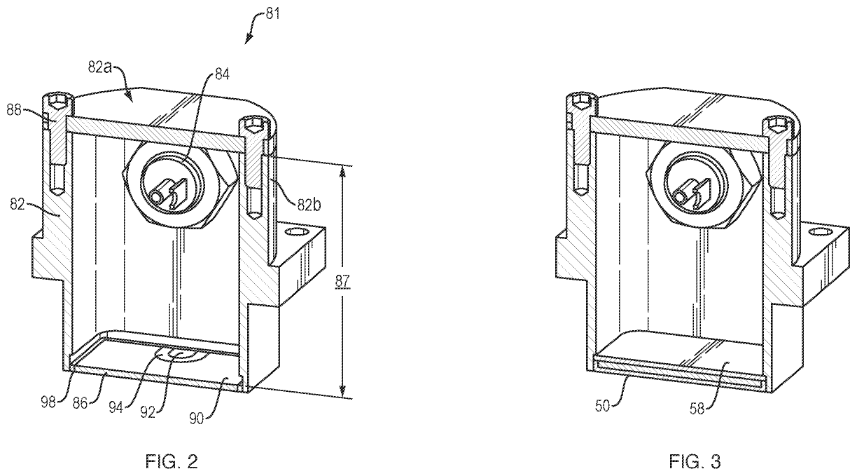

FIG. 2 is a cross-sectional view of an ultrasonic transducer 81 of the present disclosure. Transducer 81 is shaped as a disc or a plate, and has an aluminum housing 82. The piezoelectric crystal is a mass of perovskite ceramic crystals, each consisting of a small, tetravalent metal ion, usually titanium or zirconium, in a lattice of larger, divalent metal ions, usually lead or barium, and O2-ions. As an example, a PZT (lead zirconate titanate) crystal 86 defines the bottom end of the transducer, and is exposed from the exterior of the housing. The crystal is supported on its perimeter by a small elastic layer 98, e.g. silicone or similar material, located between the crystal and the housing. Put another way, no wear layer is present. In particular embodiments, the crystal is an irregular polygon, and in further embodiments is an asymmetrical irregular polygon.

Screws 88 attach an aluminum top plate 82a of the housing to the body 82b of the housing via threads. The top plate includes a connector 84 for powering the transducer. The top surface of the PZT crystal 86 is connected to a positive electrode 90 and a negative electrode 92, which are separated by an insulating material 94. The electrodes can be made from any conductive material, such as silver or nickel. Electrical power is provided to the PZT crystal 86 through the electrodes on the crystal. Note that the crystal 86 has no backing layer or epoxy layer. Put another way, there is an air gap 87 in the transducer between aluminum top plate 82a and the crystal 86 (i.e. the air gap is completely empty). A minimal backing 58 and/or wear plate 50 may be provided in some embodiments, as seen in FIG. 3.

The transducer design can affect performance of the system. A typical transducer is a layered structure with the ceramic crystal bonded to a backing layer and a wear plate. Because the transducer is loaded with the high mechanical impedance presented by the standing wave, the traditional design guidelines for wear plates, e.g., half wavelength thickness for standing wave applications or quarter wavelength thickness for radiation applications, and manufacturing methods may not be appropriate. Rather, in one embodiment of the present disclosure the transducers, there is no wear plate or backing, allowing the crystal to vibrate in one of its eigenmodes (i.e. near eigenfrequency) with a high Q-factor. The vibrating ceramic crystal/disk is directly exposed to the fluid flowing through the flow chamber.

Removing the backing (e.g. making the crystal air backed) also permits the ceramic crystal to vibrate at higher order modes of vibration with little damping (e.g. higher order modal displacement). In a transducer comprising a crystal with a backing, the crystal vibrates with a more uniform displacement, like a piston. Removing the backing allows the crystal to vibrate in a non-uniform displacement mode. The higher order the mode shape of the crystal, the more nodal lines the crystal may have. The higher order modal displacement of the crystal creates more trapping lines, although the correlation of trapping line to node is not necessarily one to one, and driving the crystal at a higher frequency will not necessarily produce more trapping lines.

In some embodiments, the crystal may have a backing that minimally affects the Q-factor of the crystal (e.g. less than 5%). The backing may be made of a substantially acoustically transparent material such as balsa wood, foam, or cork which allows the crystal to vibrate in a higher order mode shape and maintains a high Q-factor while still providing some mechanical support for the crystal. The backing layer may be a solid, or may be a lattice having holes through the layer, such that the lattice follows the nodes of the vibrating crystal in a particular higher order vibration mode, providing support at node locations while allowing the rest of the crystal to vibrate freely. The goal of the lattice work or acoustically transparent material is to provide support without lowering the Q-factor of the crystal or interfering with the excitation of a particular mode shape.

Placing the crystal in direct contact with the fluid also contributes to the high Q-factor by avoiding the dampening and energy absorption effects of the epoxy layer and the wear plate. Other embodiments may have wear plates or a wear surface to prevent the PZT, which contains lead, contacting the host fluid. This may be desirable in, for example, biological applications such as separating blood. Such applications might use a wear layer such as chrome, electrolytic nickel, or electroless nickel. Chemical vapor deposition could also be used to apply a layer of poly(p-xylylene) (e.g. Parylene) or other polymers or polymer films. Organic and biocompatible coatings such as silicone or polyurethane are also usable as a wear surface.

An ultrasonic transducer, such as ultrasonic transducer 81, for example, may be driven so that the piezoelectric material is excited in a multimode shape, which can generate multi-directional acoustic waves in a fluid to which the ultrasonic transducer is coupled. A reflector can be arranged opposite the ultrasonic transducer to reflect the multi-directional acoustic waves. The reflector can be implemented as a passive ultrasonic transducer, where the piezoelectric material reflects the incoming multi-directional acoustic wave. Alternatively, or in addition, the reflector can be implemented as an active ultrasonic transducer, where excitation of the reflective piezoelectric material provides another source for generating acoustic waves in the fluid.

The reflector may be implemented with a number of facets that are at various, potentially different distances from the ultrasonic transducer. The different distances between the reflective facets and the transducer permits a number of different resonances to be achieved when the ultrasonic transducers operating. Some or all of the facets may face the ultrasonic transducer, meaning that the reflective surface of the facets are parallel with the unexcited face of the piezoelectric material. Some or all of the facets may face away from the ultrasonic transducer, meaning that the reflective surface of the facets are not parallel with the unexcited face of the piezoelectric material. Facets that face away from the ultrasonic transducer reflect acoustic waves from the ultrasonic transducer in a direction other than directly back to the ultrasonic transducer. For example, given a line that represents the shortest distance between a facet and the unexcited face of the piezoelectric material, the reflective surface of the facet reflects an incoming acoustic wave in a direction that is at an angle with the line. Accordingly, such facets in such a reflector scatter the reflection of the incoming acoustic wave, which scattering can be in multiple, distinct, different directions. Accordingly, configuration and/or operation of the reflector permits specifically designed acoustic wave patterns, leading to an engineered acoustic field between the ultrasonic transducer and the reflector.

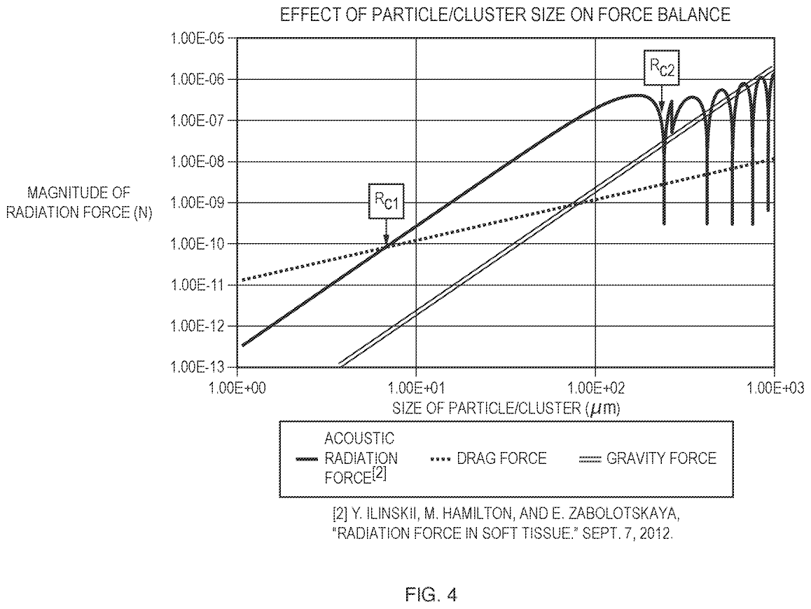

FIG. 4 is a log-log graph (logarithmic y-axis, logarithmic x-axis) that shows the scaling of the acoustic radiation force, fluid drag force, and gravity/buoyancy force with particle radius, and provides an explanation for the separation of particles using acoustic radiation forces. The gravity/buoyancy force is a particle volume dependent force, and is therefore negligible for particle sizes on the order of micron, but grows, and becomes significant for particle sizes on the order of hundreds of microns. The fluid drag force (Stokes drag force) scales linearly with fluid velocity, and therefore typically exceeds the gravity/buoyancy force for micron sized particles, but is negligible for larger sized particles on the order of hundreds of microns. The acoustic radiation force scaling is different. When the particle size is small, Gorkov's equation is accurate and the acoustic trapping force scales or is proportional to the volume, or cube of the radius, of the particle. As the size of the particle increases, the acoustic radiation force changes from being proportional with the cube of the particle radius, and rapidly vanishes at a certain critical particle size, identified in FIG. 4 as Rc2. As particle size increases beyond Rc2, the acoustic radiation force increases again in magnitude but with opposite phase (not shown in the graph). This pattern repeats for increasing particle sizes.

As a particle suspension that is composed primarily of small, single digit micron sized particles is exposed to an acoustic field, the acoustic radiation force may balance the combined effect of fluid drag force and gravity/buoyancy force. At this balanced force point, the particles may be trapped in the acoustic field. In FIG. 4, the trapping of particles smaller than about 10 μm begins to occur for particle sizes near point Rc1. As illustrated in FIG. 4, particles with sizes greater than Rc1 experience acoustic radiation force as the primary force, and are thus trapped in the acoustic field. Accordingly, as small particles, e.g., less than about 10 μm, are trapped in the acoustic field, particle clusters begin to form that increase in volume, which particle clusters experience increased acoustic radiation force, leading to increased clustering. Additionally, secondary inter-particle forces, such as Bjerknes forces, aid in particle clustering. These secondary inter-particle forces can include secondary acoustic radiation forces, where particles in an acoustic field experience an attraction toward each other. The radiation pressure generated from the acoustic scattering on particles can cause the mutual attraction or repulsion of particles. These secondary acoustic radiation forces may depend upon the resonance frequency of the particles and the distance between the particles. The particles continue to coalesce/clump/aggregate/agglomerate or cluster, resulting in continuous growth of effective particle size.

As particles cluster, the total drag on the cluster may be reduced, compared with the sum of the drag forces on the individual particles in a cluster. The clustering of the particles thus contributes to reducing net drag, since the particles in the cluster shield each other from the drag forces and reduce the comparative overall drag of the cluster.

As the particle cluster size grows, the net acoustic radiation force decreases per unit volume. However, as the acoustic radiation force on the particle continues to be primary over the drag force, the particles, and particle clusters, remain captured and trapped in the acoustic field and continue to grow in size. As particle size growth continues, the gravity/buoyancy force on the particle becomes dominant, which size is indicated in FIG. 4 by point Rc2. The gravity/buoyancy force per unit volume of the cluster remains constant with particle cluster size, since it is a function of the particle density, cluster concentration and gravity constant. Accordingly, near point Rc2 the particle cluster size increases to the point where the gravity/buoyancy force on the particle cluster increases faster than the acoustic radiation force. At the size indicated by point Rc2, the particle clusters will rise or sink, depending on their relative density with respect to the host fluid. At this size, acoustic forces have less influence, gravity/buoyancy forces become dominant, and the particles naturally drop out or rise out of the host fluid. Not all particles will drop out, and those remaining particles and new particles entering the acoustic chamber will continue to move to the three-dimensional nodal locations, repeating the growth and drop-out process. This phenomenon explains the quick drops and rises in the acoustic radiation force beyond size Rc2. Thus, FIG. 4 explains how small particles can be trapped continuously in a standing wave, grow into larger particles or clumps, and then eventually will rise or settle out because of increased gravity/buoyancy force.

The size, shape, and thickness of the transducer determine the transducer displacement at different frequencies of excitation, which in turn affects particle separation efficiency. Higher order modal displacements generate three-directional acoustic standing waves with strong gradients in the acoustic field in all directions, thereby creating equally strong acoustic radiation forces in all directions, leading to multiple trapping lines, where the number of trapping lines correlate with the particular mode shape of the transducer.

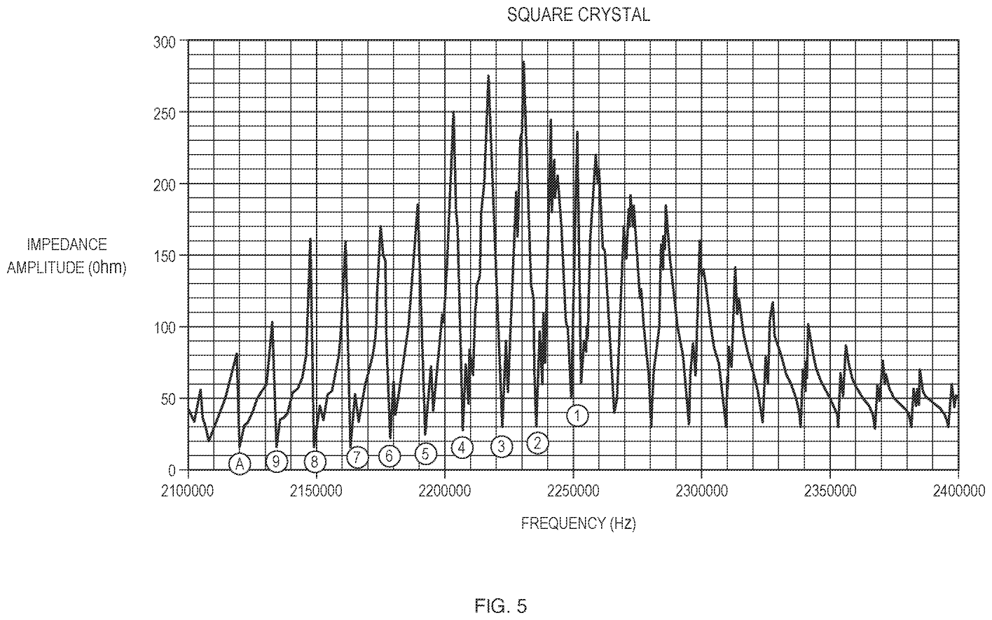

FIG. 5 shows the measured electrical impedance amplitude of the transducer as a function of frequency in the vicinity of the 2.2 MHz transducer resonance. The minima in the transducer electrical impedance correspond to acoustic resonances of a water column and represent potential frequencies for operation. Numerical modeling has indicated that the transducer displacement profile varies significantly at these acoustic resonance frequencies, and thereby directly affects the acoustic standing wave and resulting trapping force. Since the transducer operates near its thickness resonance, the displacements of the electrode surfaces are essentially out of phase. The typical displacement of the transducer electrodes is not uniform and varies depending on frequency of excitation. Higher order transducer displacement patterns result in higher trapping forces and multiple stable trapping lines for the captured particles.

To investigate the effect of the transducer displacement profile on acoustic trapping force and particle separation efficiencies, an experiment was repeated ten times, with all conditions identical except for the excitation frequency. Ten consecutive acoustic resonance frequencies, indicated by circled numbers 1-9 and letter A on FIG. 5, were used as excitation frequencies. The conditions were experiment duration of 30 min, a 1000 ppm oil concentration of approximately 5-micron SAE-30 oil droplets, a flow rate of 500 ml/min, and an applied power of 20 W.

As the emulsion passed by the transducer, the trapping lines of oil droplets were observed and characterized. The characterization involved the observation and pattern of the number of trapping lines across the fluid channel, as shown in FIG. 6, for seven of the ten resonance frequencies identified in FIG. 5.

FIG. 7 shows an isometric view of the system in which the trapping line locations are being determined. FIG. 8 is a view of the system as it appears when looking down the inlet, along arrow 814. FIG. 9 is a view of the system as it appears when looking directly at the transducer face, along arrow 816.

The effect of excitation frequency clearly determines the number of trapping lines, which vary from a single trapping line at the excitation frequency of acoustic resonance 5 and 9, to nine trapping lines for acoustic resonance frequency 4. At other excitation frequencies four or five trapping lines are observed. Different displacement profiles of the transducer can produce different (more) trapping lines in the standing waves, with more gradients in displacement profile generally creating higher trapping forces and more trapping lines. It is noted that although the different trapping line profiles shown in FIG. 6 were obtained at the frequencies shown in FIG. 5, these trapping line profiles can also be obtained at different frequencies.

FIG. 6 shows the different crystal vibration modes possible by driving the crystal to vibrate at different fundamental frequencies of vibration. The 3D mode of vibration of the crystal is carried by the acoustic standing wave across the fluid in the chamber all the way to the reflector and back. The resulting multi-directional standing wave can be thought of as containing two components. The first component is a planar out-of-plane motion component (uniform displacement across crystal surface) of the crystal that generates a standing wave, and the second component is a displacement amplitude variation with peaks and valleys occurring in both lateral directions of the crystal surface. Three-dimensional force gradients are generated by the standing wave. These three-dimensional force gradients result in lateral radiation forces that stop and trap the particles with respect to the flow by overcoming the viscous drag force. In addition, the lateral radiation forces are responsible for creating tightly packed clusters of particles. Therefore, particle separation and gravity-driven collection depends on generating a multi-directional standing wave that can overcome the particle drag force as the mixture flows through the acoustic standing wave. Multiple particle clusters are formed along trapping lines in the axial direction of the standing wave, as presented schematically in FIG. 6.

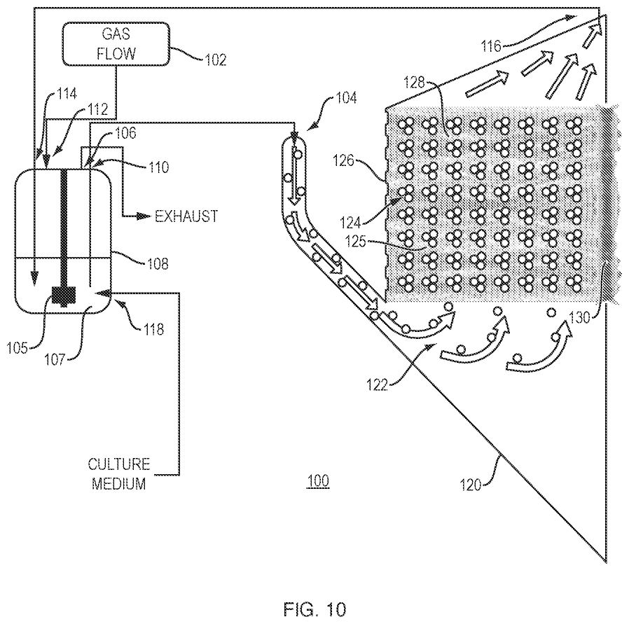

Referring to FIG. 10, a system 100 with a bioreactor 108 and an acoustic device 120 is shown. Bioreactor 108 implements a number of processes to grow cells or maintain their health. Bioreactor 108 includes a heater/impeller 105, an exhaust vent 106, an outlet 110 that is in communication with a fluid 107, an inlet 112 for receiving input gases, and an inlet 114 that represents a return path to bioreactor 108. An inlet 118 is configured to permit culture medium to be supplied to bioreactor 108.

Bioreactor 108 is fluidly connected to acoustic device 120, which connection may be a closed connection that preserves isolation of the content of bioreactor 108. For example, the connections between bioreactor 108 and acoustic device 120 may be sterilized and isolated from external communication to form a closed fluid system. Acoustic device 120 has an inlet 104 connected to bioreactor 108 via outlet 110. Inlet 104 provides a fluid pathway from bioreactor 108 to permit fluid 107, containing particles 122, to be provided to acoustic device 120. The provisioning of fluid 107 and particles 122 to acoustic device 120 may be achieved through the use of pumps and valves (not shown) between outlet 110 and inlet 104. Acoustic device 120 is provisioned with an outlet 116, which provides a return path to bioreactor 108 via inlet 114. Outlet 116 can return fluid and cells to bioreactor 108 from acoustic device 120.

Other connections, material pathways and/or outlets (not shown), each of which may or may not be fluidly closed or sterilized, may be provided to acoustic device 120 to permit alternate or additional operations. For example, outlet 116 can be connected to a fluid valve or switch to permit fluid exiting outlet 116 to be diverted to another output than bioreactor 108. In some implementations, acoustic device 120 may be configured as a concentrate/wash device that permits retained cells to be washed with a wash buffer or subject to a media exchange. The fluid removed in such an operation may be taken from outlet 116 and diverted to a waste container. The fluid input to acoustic device 120 in such an operation may be supplied to an inlet located at a lower portion of acoustic device 120. Other connections, material pathways and/or outlets can provide different materials to different devices or receptacles, such as by providing a cell product via an outlet (not shown) at a lower portion of acoustic device 120, or a supernatant outlet (not shown) to draw off fluid or material at particular locations or levels in acoustic device 120.

Acoustic device 120 includes an ultrasonic transducer 130, which may be implemented as transducer 81 (FIG. 2). Ultrasonic transducer 130 may be implemented as a number of transducers in close proximity, or may be implemented as an array of transducers formed from a single piezoelectric crystal. As illustrated in FIG. 10, ultrasonic transducer 130 is configured and operated to deform to have a wave shape across its surface. Three peaks are visible on each side of ultrasonic transducer 130, illustrating the multimode operation in a 3×3 mode (3,3). In this 3×3 multimode, ultrasonic transducer 130 has a displacement profile similar to that of number 4 illustrated in FIG. 6. Ultrasonic transducer 130 launches acoustic waves in multiple directions in an acoustic volume 125 due to the deformation of ultrasonic transducer 130 in multimode operation. As discussed above, the multi-directional acoustic wave contributes to formation of an acoustic field 128 that has locales of higher and lower pressures and/or acoustic radiation forces that act on particles 122 in the field to cause particle clustering, resulting in particle clusters 124. As fluid 107 and particles 122 are passed from bioreactor 1082 acoustic device 120, particles 122 are trapped into particle clusters 124 by acoustic field 128. Fluid 107 passes through acoustic field 128 to outlet 116, and returns to bioreactor 108 via inlet 114. Particle clusters 124 may shift within acoustic volume 125 as the locales of low pressure in acoustic volume 125 shift with changes in acoustic field 128. As discussed above, particle clusters 124 may reach a critical dimension where they exit acoustic volume 128, and may collect in a collection volume below acoustic volume 128. The flow of fluid 107 and particles 122 into acoustic device 120 may be modulated to permit particles 122 to have a concentration that maintains particle clusters 124 in acoustic volume 125. For example, the number, concentration and/or volume of particles 122 flowed into acoustic device 120 can be controlled so that particles 120 to are retained as particle clusters 124 in acoustic volume 125, without a significant amount of escaping particles. Control of the flow of fluid 107 and particles 122 may be modulated with pumps and flowmeters in conjunction with a controller. The operation of ultrasonic transducer 130 may also be controlled to further contribute to maintaining particle clusters 124 in acoustic volume 125, such as by using a controller to modulate power and/or frequency of the drive signal delivered to ultrasonic transducer 130. Flow control, as well as transducer drive control can be provided through the use of a controller, in conjunction with pumps, valves and meters, as discussed above.

A non-planar reflector 126 is provided opposite to ultrasonic transducer 130. Reflector 126 reflects acoustic energy, including acoustic waves, at a number of different resonances and/or in a number of different directions. Reflector 126 can be implemented with a number of distinct facets that face a number of different directions to scatter the incoming acoustic waves or acoustic energy. In addition, or alternatively, reflector 126 can include a number of distinct facets that face and are each different distances from ultrasonic transducer 130 so that a number of different resonances are possible between reflector 126 and ultrasonic transducer 130.

Reflector 126 may be configured and designed to reflect acoustic energy in certain patterns to form an engineered acoustic field. For example, reflector 126 may disrupt resonance within acoustic volume 125 at one or more operating frequencies or multimodes. In some circumstances, reflector 126 may contribute to disruption of a standing wave in acoustic volume 125, such that no acoustic standing wave is established. In some implementations, reflector 126 may contribute to formation of standing waves in acoustic volume 125. Ultrasonic transducer 130 can be configured and operated to generate multi-directional acoustic waves, which are reflected by reflector 126 to form a wave interference pattern that results in acoustic field 128. As illustrated, acoustic field 128 includes locales of low pressure, to which pressure and/or acoustic radiation forces drive particles to permit particles to be trapped and clustered to form larger effective particles.

Acoustic device 120 may be implemented with other systems and in other forms. For example, acoustic device 120 may receive an input from a closed container, such as a bag containing patient blood or blood components, and may obtain input from other sources, such as a wash medium source or reagent source. Acoustic device 120 may be implemented to output material to various vessels other than bioreactor 108. For example, acoustic device 120 may be connected to a waste bag for outputting spent media or exchanged media. An outlet can connect acoustic device 120 to a product bag or container for containing a product. Acoustic device 120 may be connected to multiple sources and/or multiple output devices or receptacles. For example, acoustic device 120 may be connected to bioreactor 108 for input, and provide a product output to another device or receptacle, in addition to, or alternatively to, providing a return output to bioreactor 108.

In accordance with the present disclosure, acoustic field 128 captures and retains particles 122 to form particle clusters 124. As noted elsewhere herein, particles 122 can be beads, cells or cell-bead complexes. Particle clusters 124 act as seeding particles by producing secondary acoustic radiation forces that act on nearby bodies to produce attraction between particle clusters 124 and the bodies. The secondary acoustic radiation forces tend to draw particles together, even when the separated particles are of uneven size.

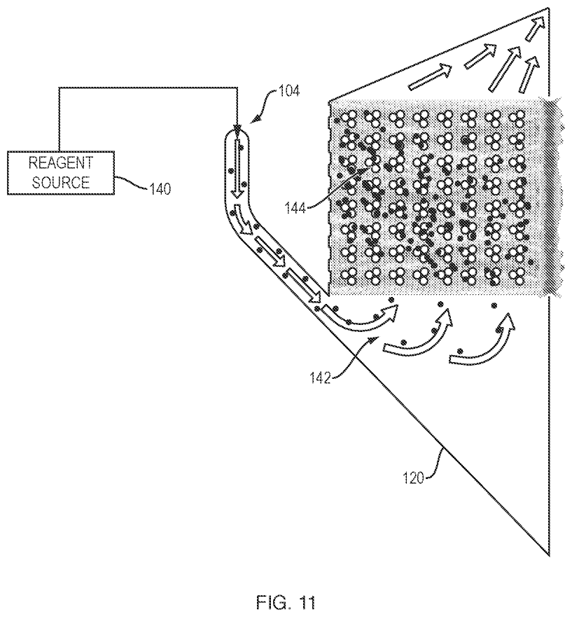

Referring also to FIG. 11, a reagent source 140 is fluidly connected to acoustic device 120. Although reagent source 140 is shown connected to inlet 104, other input connections are possible, such as at outlet 116 (FIG. 10). Reagent source 140 supplies reagent 142 to acoustic device 120, which supply can be via a close, sterile fluid connection. Reagent 142 is composed of particles that may be in the form of beads, droplets, viruses, oligomers, including oligonucleotides, molecules, or any other structure with a size in the micro range or smaller. Reagent 142 is flowed into the acoustic device 120, along a fluid path that is directed to acoustic volume 125. Acoustic volume 125 has been preloaded with particles 122, which are maintained in acoustic volume 125 by acoustic field 128. Upon entering acoustic volume 125, reagent 142 is driven to particle clusters 124. Reagent 142 may be driven to particle clusters 124 due to pressure and/or acoustic radiation forces, and/or due to secondary acoustic radiation forces and/or due to kinetic forces between bodies. In particular, secondary acoustic radiation forces create a mutual attraction between particles of reagent 142 and particle clusters 124. Particle clusters 124 are maintained in acoustic field 128 by primary acoustic radiation forces, which forces have less of an effect on the particles of reagent 142 due to their smaller size. Secondary acoustic radiation forces do have a significant influence on the particles of reagent 142, and urge those particles toward particle clusters 124. Although they also experience secondary acoustic radiation forces, particle clusters 124 do not leave the locales of the acoustic field in which they are trapped. According, the particles of reagent 142 tend to move towards particle clusters 124 due to the influence of secondary acoustic radiation forces. Thus, the likelihood of interaction 144 between particle clusters 124 and reagent 142 is increased within acoustic volume 125 due to the effects of acoustic field 128. The fluid in which reagent 142 is entrained flows through the acoustic field 128 and exits acoustic device 120 via outlet 116.

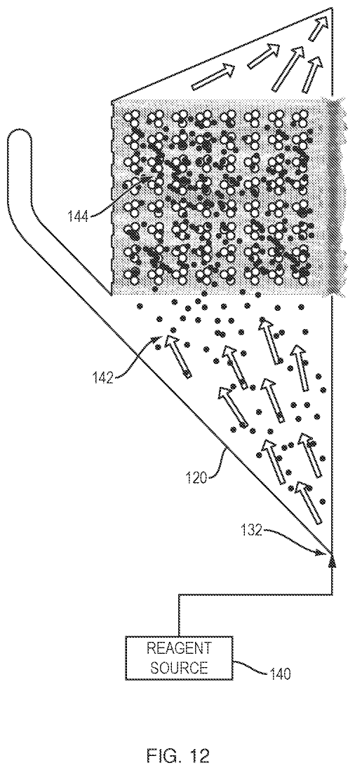

Referring also to FIG. 12, a configuration is shown where reagent source 140 supplies reagent 142 via an inlet 132 of acoustic device 120. The geometry of acoustic device 120 can permit reagent 142 to be diffused throughout a fluid volume prior to entering acoustic volume 125. Flowing reagent 142 through inlet 132 to improve diffusion of reagent 142 may lead to improved interaction 144 and acoustic volume 125.

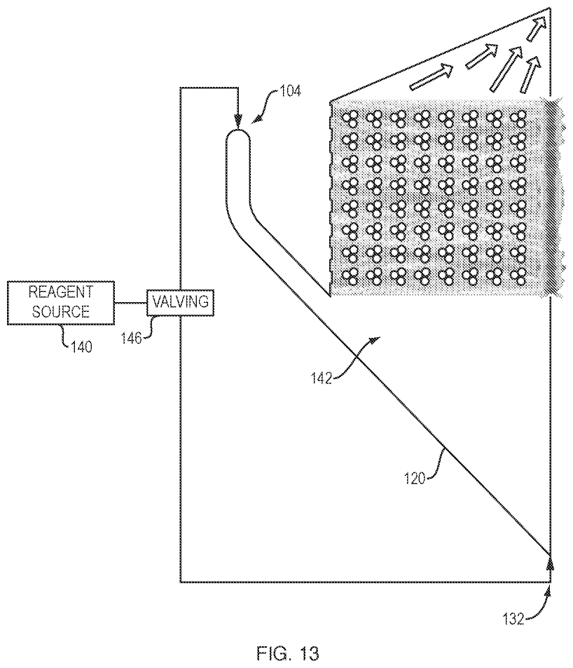

Referring also to FIG. 13, a configuration is shown where reagent source 140 supplies reagent 142 through one or more of inlets 104, 132 in accordance with valving 146. By permitting reagent 142 to flow into acoustic device 120 via multiple inlets, different reagent modes can be realized. For example, particle clusters 124 may be composed of beads in the range of 10-50 μm, and two different reagents may be supplied to acoustic device 120 through the different inlets 104, 132. The two different reagents can be driven to interact with each other due to one or more of pressure, acoustic radiation force, secondary acoustic radiation force and/or kinetic forces. As another example, a wash buffer or secondary media may be supplied from reagent source 140 to inlet 132 via valving 146. Another reagent 142, such as functionalized beads, T-cell activation reagent, viruses or other compounds may be supplied from reagent source 140 to inlet 104 via valving 146. The wash buffer or secondary media may be supplied at the same time, or at different times than reagent 142. Accordingly, acoustic device 120 permits interaction between a number of different materials within acoustic volume 125, which materials may be variously supplied from external sources, including bioreactor 108, reagent source 140, or such sources as bag for containing patient blood or blood products.

Other connections, material pathways and/or outlets (not shown), each of which may or may not be fluidly closed or sterilized, may be provided to acoustic device 120 to permit alternate or additional operations. For example, outlet 116 can be connected to a fluid valve or switch to permit fluid exiting outlet 116 to be diverted to different outputs, including bioreactor 108 and/or a waste container. In some implementations, acoustic device 120 may be configured as a concentrate/wash device that permits retained cells to be washed with a wash buffer or subject to a media exchange. The fluid removed in such an operation may be taken from outlet 116 and diverted to a waste container. The fluid input to acoustic device 120 in such an operation may be supplied to inlet 104 or 132. Other connections, material pathways and/or outlets can provide different materials to different devices or receptacles, such as by connecting inlet 132 to a cell product receptacle so that inlet 132 can be used as an outlet for a cell product. An outlet (not shown) may be provided between inlet 132 and acoustic volume 128 to draw off material, including a supernatant. Other outlets and/or connections to draw off fluid or material at particular locations or levels in acoustic device 120 may also be provided.

In an example implementation, T cells are supplied to acoustic device 120 from a source, such as bioreactor 108 or a patient blood bag. The T cells may be unaltered patient T cells, or CAR-T cells as discussed above. The T cells are clustered in acoustic field 128 to form T cell clusters, such as is illustrated with particle clusters 124. As the T cells are clustered together, they can be activated in a self-catalytic process where the T cells produce IL-2 that is delivered to the neighboring T cells in the cluster. This self-catalytic process is sometimes referred to as the paracrine effect.

T cells that are clustered in an acoustic field such as acoustic field 128 can be supplied with high or low molecular weight (MW) reagents. For example, activation, transduction or transfection operations may use high MW reagents, such as functionalized beads, viruses, plasmids or other materials that may be less than 50 nm in size. Such operations may employ slow diffusion processes of the reagents in the T cell suspension. The diffusion processes may use excess concentrations of reagent, and/or process time to implement the operation, leading to greater expense involving costly reagents, and greater length of time to complete the operation. When supplied to acoustic device 120, high MW reagents can interact with T cells that are captured within an entire acoustic volume 125, where the reagents are driven to the T cell clusters to improve interaction between the reagents and the T cells. With this approach, a reduced amount of reagent may be employed to achieve the operation, leading to reduced expense for the costly reagent. In addition, the high MW reagent is more purposefully and efficiently driven to interact with the T cells, thereby reducing the time for completing the operation.

By seeding acoustic field 128 with T cells that are on the order of tens of microns, more efficient interaction with high MW reagents that are on the order of 50 nm or less can be achieved. When known diffusion methods are used, high MW reagents take longer to reach the surface of the T cell than low MW reagents, leading to longer incubation times and usage of greater concentrations of high MW reagents to achieve the operation. When high MW reagents are provided to an acoustic field that captures and maintains T cells, such as acoustic field 128, secondary acoustic forces obtained from T cell clustering can drive high MW reagents to the T cell clusters to enhance interaction. Secondary acoustic forces obtained from T cell clustering can enhance fluid viscosity around the T cell cluster, further aiding in reagent trapping in the locale of the T cell cluster and contributing to limiting reagent wash out from the acoustic field.

Interactions between different materials within acoustic volume 125 may be achieved according to a number of different modes, some of which are discussed above. According to another example implementation, particles with dimensions, such as diameters, on the order of tens of microns may be provided to acoustic volume 125 and captured within acoustic field 128 to form particle clusters 124. In the interaction mode of this example, the particles may be cells, or cell and bead complexes. A reagent 142 may be introduced to acoustic volume 125, which reagent 142 may be composed of degradable beads such as agarose beads that are sized on the order of tens of microns. The beads may be configured to have a same contrast factor as the particles captured in acoustic field 128. The beads may be functionalized with reagent material, such as cell activation material, viruses or plasmids, as examples, which tend to be sized on the order of less than 50 nm, and also tend to be highly expensive, which can significantly drive up the cost of cell processing. With the functionalized, degradable beads as reagent 142, introduction of reagent 142 two acoustic volume 125 obtains a much greater capturing and clustering effect than the reagent material alone since the beads of reagent 142 are clustered with the primary pressure and/or acoustic radiation forces of acoustic field 128. Accordingly, reagent 142 is clustered together more readily with particle clusters 124, creating significant interaction between cells and the reagent material. The amount of reagent material used to functionalize the beads of reagent 142 can be reduced, even an order of magnitude, since no reagent material needs to be provided to areas outside of the locales of particle clusters 124.

Other modes of operation where seeding particles are provided to an acoustic field such as acoustic field 128 are possible. For example, it may be desirable to filter particles out of the fluid that are 2-3 μm or smaller in size. Acoustic filtering with transducers that operate in the 0.5-10 MHz range may be implemented effectively for particles in the range of tens to hundreds of microns in size, as illustrated in FIG. 4. Above the size indicated by point Rc1, for example, acoustic radiation forces are dominant over drag and gravity/buoyancy forces, permitting particles in that size range to be captured and retained in the acoustic field against the drag force of the fluid in which they are entrained. Particles in the size range below Rd are primarily affected by drag force and tend not to be captured when the fluid in which they are entrained is flowed through the acoustic field. Accordingly, using an acoustic field to capture and filter particles that are less than about 10 μm in size can be challenging. By providing in the fluid particles that are in the range of tens to hundreds of microns in size, which are readily captured in the acoustic field, that is, by seeding the acoustic field, smaller sized particles can be effectively captured and filtered.

According to an example implementation, particles entrained in a fluid are provided to an acoustic field, such as acoustic field 128, where the size of the particles is in the range of tens to hundreds of microns. The particles may be PMMA beads, polystyrene beads, glass beads, perfluorocarbon beads, including perfluorohexane beads, or any other kind of bead that has a size distribution in the range of tens to hundreds of microns and an acoustic contrast factor with a same sign (positive or negative) as that of the smaller-sized target material to be captured and filtered. The particles are captured in the acoustic field, with some of the particles being clustered at locales of lower pressure and/or acoustic radiation force. The smaller-sized target material is provided to the acoustic field, such as by flowing a fluid in which the target material is entrained to the acoustic field. As the target material enters the acoustic field, the particles and clustered particles exert a secondary acoustic radiation force on the target material, in addition to the primary acoustic radiation force being exerted by the acoustic field. The net force drives the target material to the particles and clustered particles captured in the acoustic field, to effectively capture the target material in the locales occupied by the particles and clustered particles. The targeted material may thus be captured in the acoustic field and retained against drag forces through the use of seeding particles provided to the acoustic field.

An example implementation of the above discussed technique was applied to platelet cells in a patient's blood. The size of platelet cells is on the order of 2-3 μm at their largest diameter, and are not easily captured in an acoustic field as discussed herein. Separating platelet cells from a patient's blood is desired for implementing CAR-T cell processing, and has been previously implemented using centrifugation. Centrifugation can generate high shear forces on the cells in the patient's blood, leading to necrosis (cell death). Through the seeding of an acoustic field, as discussed herein, platelet cells can be captured and filtered from a patient's blood using acoustic separation techniques, without imposing significant shear forces so as to avoid mechanistic necrosis.

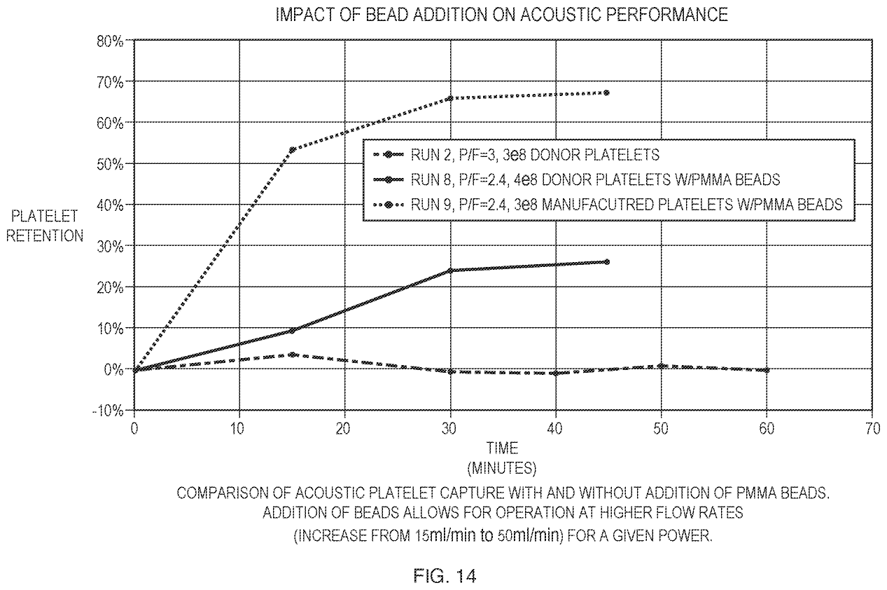

FIG. 14 is a graph that illustrates the results of a particle retention technique where seeding particles are provided to an acoustic field to contribute to retaining platelets in the acoustic field. In the example results illustrated in FIG. 14, PMMA beads were used as the seeding particles. The contrast factor of the seeding particles is similar to that of the target material, in this example, platelets, in that the sign (positive or negative) is the same and the magnitude is greater or relatively similar to the target material. The power provided to the ultrasonic transducer contributes to acoustic field strength and was maintained constant over the runs illustrated in FIG. 14. The graph in FIG. 14 illustrates an increase in platelet capture with the addition of PMMA beads. For example, Run 8, where 4×10⁸ donor platelets were provided to the acoustic field with PMMA beads, improved platelet retention from about 0% to about 25% after 30 minutes. Run 9, where 3×10⁸ manufactured platelets were provided to the acoustic field with PMMA beads, improved platelet retention from about 0% to about 65% after 30 minutes.

Based on these improved platelet retention rates with the use of seeding particles, acoustic separation of platelets from a fluid in which they are entrained can be achieved at higher flow rates. For example, as noted in FIG. 14, the flowrate of platelet-containing-fluid that is passed through the seeded acoustic field while retaining platelets in the acoustic field can be increased from 15 ml/min to 50 ml/min for a given power, a 333% increase in throughput. This result is significant, considering that the percentage of platelet retention is close to 0% when seeding particles are not present in the acoustic field, as illustrated by the results for Run 2, where 3×10⁸ donor platelets are presented to the acoustic field. According to this result, flow rate has no impact on platelet retention in the absence of seeding particles, since substantially all of the platelets pass through the acoustic field without retention.

The high (primary) acoustic radiation force on the seeding particles causes them to be trapped in the acoustic field, and permits them to exert secondary acoustic radiation forces on the target material provided to the acoustic field. The combination of primary & secondary acoustic forces increases the net acoustic force experienced by the target particles and permits them to be retained in the acoustic field against drag force or gravity/buoyancy force. The frequency as well as the power of the drive signal applied to the ultrasonic transducer can be specified for the size of the seeding particles to retain them in the acoustic field. The amount and/or concentration of the seeding particles can be specified to permit them to occupy and be retained in the acoustic volume in which the acoustic field is formed so as to avoid settling or otherwise leaving the acoustic field, e.g. via fluid flow. By increasing the force on the target material in the acoustic field with the seeding particles, the target material can be trapped, and the fluid velocities can be significantly increased, permitting greater yields in a shorter amount of time.

With the use of seeding particles, increased throughput in acoustic concentrate wash devices can be achieved. The seeding represents an enabling tool for capture of very dilute and/or very small/low contrast materials in an acoustic field. FIG. 14 illustrates a comparison of platelet capture with and without the addition of PMMA beads. Addition of beads allows for operation at higher flow rates (increase from 15 ml/min to 50 ml/min) for a given power.

The methods, systems, and devices discussed above are examples. Various configurations may omit, substitute, or add various procedures or components as appropriate. For instance, in alternative configurations, the methods may be performed in an order different from that described, and that various steps may be added, omitted, or combined. Also, features described with respect to certain configurations may be combined in various other configurations. Different aspects and elements of the configurations may be combined in a similar manner. Also, technology evolves and, thus, many of the elements are examples and do not limit the scope of the disclosure or claims.

Specific details are given in the description to provide a thorough understanding of example configurations (including implementations). However, configurations may be practiced without these specific details. For example, well-known processes, structures, and techniques have been shown without unnecessary detail to avoid obscuring the configurations. This description provides example configurations only, and does not limit the scope, applicability, or configurations of the claims. Rather, the preceding description of the configurations provides a description for implementing described techniques. Various changes may be made in the function and arrangement of elements without departing from the spirit or scope of the disclosure.

Also, configurations may be described as a process that is depicted as a flow diagram or block diagram. Although each may describe the operations as a sequential process, many of the operations can be performed in parallel or concurrently. In addition, the order of the operations may be rearranged. A process may have additional stages or functions not included in the figure.

Having described several example configurations, various modifications, alternative constructions, and equivalents may be used without departing from the spirit of the disclosure. For example, the above elements may be components of a larger system, wherein other structures or processes may take precedence over or otherwise modify the application of the invention. Also, a number of operations may be undertaken before, during, or after the above elements are considered. Accordingly, the above description does not bound the scope of the claims.

A statement that a value exceeds (or is more than) a first threshold value is equivalent to a statement that the value meets or exceeds a second threshold value that is slightly greater than the first threshold value, e.g., the second threshold value being one value higher than the first threshold value in the resolution of a relevant system. A statement that a value is less than (or is within) a first threshold value is equivalent to a statement that the value is less than or equal to a second threshold value that is slightly lower than the first threshold value, e.g., the second threshold value being one value lower than the first threshold value in the resolution of the relevant system.

The present disclosure has been described with reference to exemplary embodiments. Modifications and alterations may occur to others upon reading and understanding the preceding detailed description. It is intended that the present disclosure be construed as including all such modifications and alterations insofar as they come within the scope of the appended claims or the equivalents thereof.

1. A method for capturing particles, comprising:
generating an acoustic field in a fluid chamber using an acoustophoretic device, the acoustophoretic device comprising:
an ultrasonic transducer coupled to the fluid chamber and including a piezoelectric material that is configured to deform in a multimode shape; and
exciting the ultrasonic transducer to generate a multi-directional acoustic wave in the fluid chamber to establish the acoustic field, the acoustic field including locales of greater and lesser acoustic radiation force;
receiving first particles in the fluid chamber;
trapping and retaining the first particles in the acoustic field at the locales of greater acoustic radiation force or lesser acoustic radiation force;
receiving second particles in the fluid chamber; and
trapping and retaining the second particles at the locales occupied by the first particles with a secondary acoustic radiation force.

2. The method of claim 1, wherein the sign of the acoustic contrast factor of the first particles is the same as that of the second particles.

3. The method of claim 1, wherein the size of the first particles is greater than or equal to 10 μm and the size of the second particles is less than 10 μm.

4. The method of claim 1, wherein the second particles are platelets.

5. The method of claim 1, wherein the first particles are beads.

6. The method of claim 1, further comprising reflecting the multi-directional acoustic wave using a reflector in the acoustophoretic device, wherein the reflector is arranged opposite to the ultrasonic transducer across the fluid chamber.

7. The method of claim 6, wherein the reflector includes a surface facing the ultrasonic transducer, the surface including a number of facets.

8. The method of claim 7, wherein at least some facets are different distances from the ultrasonic transducer and face the ultrasonic transducer.

9. The method of claim 7, wherein at least some facets face away from the ultrasonic transducer.

10. An acoustophoretic system for capturing particles, comprising:
a fluid chamber;
an ultrasonic transducer coupled to the fluid chamber and including a piezoelectric material that is configured to deform in a multimode shape;
the ultrasonic transducer being arranged to be excited to generate a multi-directional acoustic wave in the fluid chamber to establish an acoustic field that includes locales of greater and lesser acoustic radiation force;
an inlet to the fluid chamber arranged to permit an input particle-fluid mixture to be received by the acoustic field;
wherein the particle-fluid mixture includes particles that are configured to generate secondary acoustic radiation forces when captured in the locales of greater or lesser acoustic radiation force in the acoustic field.

11. The system of claim 10, wherein the size of the particles is greater than or equal to 10 μm.

12. The system of claim 10, further comprising a reflector opposite to the ultrasonic transducer across the fluid chamber.

13. The system of claim 12, further comprising a surface of the reflector facing the ultrasonic transducer, the surface including a number of facets.

14. The system of claim 13, further comprising at least some of the facets being arranged to face the ultrasonic transducer and at different distances from the ultrasonic transducer.

15. The system of claim 13, further comprising at least some of the facets being arranged to face away from the ultrasonic transducer.

16. A method for capturing small particles less than 10 μm in size in an acoustic field, comprising:
generating an acoustic field in a fluid chamber using an acoustophoretic device, the acoustophoretic device comprising:
an ultrasonic transducer coupled to the fluid chamber and including a piezoelectric material that is configured to deform in a multimode shape; and
exciting the ultrasonic transducer to generate a multi-directional acoustic wave in the fluid chamber to establish the acoustic field, the acoustic field including locales of greater and lesser acoustic radiation force;
receiving seeding particles in the fluid chamber, where the size of the seeding particles is 10 μm or greater;
trapping and retaining the seeding particles in the acoustic field at the locales of greater acoustic radiation force or lesser acoustic radiation force;
receiving the small particles in the fluid chamber; and
trapping and retaining the small particles at the locales occupied by the seeding particles with a secondary acoustic radiation force.

17. The method of claim 16, wherein the small particles are platelets.

18. The method of claim 16, wherein the sign of the acoustic contrast factor for the small particles is the same as that of the seeding particles.

19. The method of claim 16, further comprising reflecting the multi-dimensional acoustic wave using a faceted reflector in the acoustophoretic device, wherein the faceted reflector is arranged opposite to the ultrasonic transducer across the fluid chamber.

20. The method of claim 16, further comprising receiving the small particles in the fluid chamber at a rate of greater than 15 ml/min.
